(19) United States
(12) Patent Application Publication
(10) Pub. No.: US 2007/0207190 A1
(43) Pub. Date: Sep. 6, 2007

(54) Process for manufacturing hydrophobic polymers

(75) Inventors: William Dierickx, Destelbergen (BE); Dirk De Cuyper, Heusden (BE); Yvan Dierickx, Heusden (BE); Dirk Mertens, Wettern Ten Ede (BE); Johan Mertens, Wetteren_ten Ede (BE); Lynda Beladjal, Wetteren_ten Ede (BE)

Correspondence Address: BIRCH STEWART KOLASCH & BIRCH, PO BOX 747, FALLS CHURCH, VA, 22040-0747, US

(73) Assignee: RESILUX, Wetteren (BE)

(21) Appl. No.: 11/714,385

(22) Filed: Mar. 5, 2007

Related U.S. Application Data
(63) Continuation-in-part of application No. PCT/BE05/00133, filed on Sep. 2, 2005.

(30) Foreign Application Priority Data
Sep. 3, 2004 (BE) BE 2004/0431

(51) Int. Cl. A61K 35/12; A61K 9/70

(52) U.S. Cl. 424/443; 424/93.7

(57) ABSTRACT

Method for producing hydrophobic polymers, wherein a hydrophobic polymer is selected; a set of organisms is selected from among cells and/or cell products; aggregates are formed by working said cells and/or cell products in said polymers resulting in the formation of a so-called polymer-bio aggregate, wherein there is performed a new function of the thus polymer product. This invention further relates to the use of a so-called P.B.A. obtained therewith in specific applications.

### FIELD OF THE INVENTION

The present invention relates to a method for producing polymers incorporating living organisms and/or cell products.

### PRIOR ART

This type of method is known for non-permanent, biodegradable hydrophilic polymers with a melting point well below 100° C., in which temperature-sensitive tissue cells and organic molecules are incorporated. The polymer degrades after a short time.

U.S. Pat. No. 5,985,354 in the name of MATHIOWITZ describes a method of the above type.

The problem encountered in the known prior art for the incorporation of living material is that the melting point of permanent, non-biodegradable polymers is well above 100° C. at normal pressure conditions. The implantation of living, active organisms or microorganisms at temperatures of this level is impossible without fatal consequences for these organisms. Accordingly, it has to be assumed that the incorporation of living material during the production of a usable object from a base material such as polymer is not achievable, even in case the organisms which are introduced can subsequently perform useful activity at normal ambient temperatures. Possible activities in this respect are oxygen consumption or absorption, absorption of radiant energy, including what is known as “UV blocking”, and the like. The above therefore demonstrates that currently there are considerable restrictions in the possible range of applications for living cells in this type of polymer.

JP 2002274521 discloses a container made from biodegradable polymer of a micro-organism including a coating layer of diamond like carbon/silicon dioxide, with formation of silicon carbide on the main body of the container aiming at preventing permeation of gas/water content to the inner surface of said container.

However, said document lacks in providing a suitable working temperature range susceptible or being able to solve the problem indicated above. In addition, said document merely addresses polymers of a micro-organism without specifying the specific structural link between the latter, which appears to be a laminate structure. Moreover, an isolating barrier function is indicated to be ensured by said coating layer of diamond like carbon/silicone dioxide. Consequently, said micro-organisms, which are presented equally as said chemo-synthesis group, are not fully exploited for their potential barrier properties.

EP 1 375 616 A of KIRIN BEER merely discloses working temperature ranges below 100° C. with the consequence that it lacks in providing a suitable working temperature range able to solve the problem indicated above.

As to U.S. Pat. No. 5,618,412 A of HERDING et al. it discloses a fixed-bed bioreactor for purifying fluids with the aid of micro-organisms wherein carrier bodies have a porous structure with pores adapted to be penetrated by the fluid and to have micro-organisms attached thereto. This document therefore addresses a different field, which results in the proposed structure being presented with a passing action consisting in a purifying effect of passing fluid. This is in opposition to the present invention in which the aim is to block the passage so that there is a functional antagonism between the teaching of said document intended for purifying fluids passing through the proposed device in opposition to the present invention the main aim whereof is to block the passage of undesired fluids, more particularly gasses.

A further problem encountered in the known prior art is that the slow diffusion of cellular components and biomolecules in a moist environment is based on a technology which is predicated on multi-wall microcapsules of hydrophilic, soluble or biodegradable polymers. However, there is currently no available technology which permits the slow diffusion of a gamma to biomolecules from a permanent polymeric carrier without it being degraded in an aqueous and/or dry environment. This applies in particular to biomolecules of the fatty acid type, such as lipids and hydrocarbons.

Therefore, the known prior art mainly has the following shortcoming, namely that bio-encapsulation of cells in polymers is not possible above 100° C. under standard pressure conditions, on the one hand, and that slow diffusion of cellular metabolites and related organic molecules from polymers is not possible without degradation of the polymer in a moist environment, on the other hand.

### OBJECT OF THE INVENTION

It is an object of the present invention to offer a solution to the abovementioned drawbacks and/or shortcomings.

### SUMMARY OF THE INVENTION

According to the present invention, there is proposed a method for producing polymers in which a polymer is selected, and furthermore a set of organisms is selected from cells and/or cell products, wherein aggregates are then formed by working the said cells into the said polymer, resulting in the formation of what is known as a polymer-bio aggregate, referred to below as PBA, producing a novel function of the polymer product formed in this way. Remarkably, the work is carried out in the operating temperature range taken from the temperature range for which the lower limit is set at virtually 100° C. under virtually standard pressure conditions, in particular at virtually one atmosphere.

According to a preferred embodiment of the invention, there is defined that the abovementioned cells are selected from the category of what are known as the cysts and/or in a phase of inactive or dormant stages. A quite significant number of types of organisms or microorganisms can change from an active life form to what is known as a quiescent stage or spore, known as cysts. Said spores are able to withstand extreme environmental fluctuations in a latent form. In this state of anabiosis, they are able to withstand extremely dry conditions and temperatures well above 100 °C.

Under suitable biotechnology conditions, these types can not only be cultivated but also converted, in controlled culture conditions, known an encysting, into usable spores for bio-encapsulation in a polymeric matrix.

During the production process of an industrial product, such as packaging material, textile fibers, granules or the like, said spores and the polymer are agglomerated within a short period of time during which the polymer is liquid, namely at a temperature above its melting point. This produces what is known as a polymer-bio aggregate, referred to below as “PBA”.

As long as the product is not in use, the organisms of the bio-component in said PBA remain inactive. However, as soon as the living conditions become favorable, coinciding with the product starting to be used in association with an environment which is suitable for life in terms of temperature and relative humidity, the spores change into active, metabolizing cells under these favorable ambient conditions. For this period, the biologically active form will perform its intended function. As soon as the optimum conditions revert to conditions which are less than optimum, the active form returns to the spore.

The process remains reversible in accordance with a feedback mechanism which is controlled by the living environment of the organism in said PBA.

Thus, according to a particularly preferred embodiment of the invention, said cell products are selected from the category of so-called metabolites, i.e. the molecules which are biochemically synthesized by organisms under the abovementioned temperature working conditions.

According to yet another preferred embodiment of the present invention, the polymers are selected from non-biodegradable polymers. Reliable, slow and prolonged diffusion of organic molecules out of polymers into a moist or fluctuating environment can be realized without degradation of the polymer.

An advantage obtained by virtue of the method defined by the present invention is mainly that the biological activity of the organisms incorporated in the so-called PBA produced in accordance with the invention imparts novel, previously unknown properties to the polymer. Said PBA hereby ensures the desired environment for which said PBA was made.

A further advantage consists in the standardized release of very specific biomolecules, such as so-called repellents, from a polymeric matrix, such as granules, textile fibers and the like, without said polymeric matrix being lost in a variable environment as a result of climatological instabilities, for example.

Further features and properties of the present invention are defined in further sub-claims.

Further details and particularities will emerge from the following description of a number of exemplary embodiments of the method according to the invention and its uses.

### DESCRIPTION

In general terms, the present invention relates to a method for producing polymers which incorporate cells, living organisms and/or cell products. A number of specific use examples are described below.

A particularly significant application area is in the food packaging sector which employs what is known as an oxygen barrier, with a PBA layer arranged as an intermediate layer in multi-layer packaging material for foodstuffs, such as PET bottles for beverages, such as beers or fruit juices for example. The polymer component of the PBA is in this case PET, while the PBA bio component is a type of yeast with a dry spore, such as for example Saccharomyces, which is able to withstand the high temperatures of the production process. The PBA remains inactive until the PET bottle has been filled. When the package is being filled with fruit juices or beer, for example, the internal environment of the PBA becomes water-saturated, with the result that the spores are activated to form respiring cells which consume all the oxygen present inside the bottle. As a result, all the oxygen is withdrawn from the contents under the influence of what is known as the O₂ scavenger. Also, all the external oxygen which can diffuse through the wall is captured by the yeast cells for respiration, which results in an efficient oxygen barrier.

A further example of a use consists in the action as a UV blocker, which works in a similar way to the above example. In this example, instead of the yeast cells, there is incorporated in the PBA a type of alga, such as for example Haematococcus, the spores of which very intensively block UV light. A continuous layer of Haematococcus cells, haematocysts with a high concentration of astaxanthin, makes the PBA opaque to UV light. This fact is utilized in moisture-resistant UV-proof films and polymer coverings.

A still further use consists in the combined application of both examples mentioned above in connection with food packaging with an oxygen barrier and a UV blocker which is suitable for PET bottles as packaging for beers and fruit juices and the like. The PBA biocomponent is a calibrated mixture of Saccharomyces and Haematococcus. Said oxygen scavengers, such as yeast cells for example, represent a permanent oxygen barrier, while the UV blocker, such as a type of alga, for example, prevents photochemical degradation of the filling.

Yet another application consists in the absorption of energy from sunlight with a cooling effect which is similar to the example above relating to the so-called UV blocker. Instead of Haematococcus, the PBA incorporates a type of alga such as for example Chlorococcus, the active form of which, in the presence of a high degree of moisture, participates very intensively in photosynthesis, consuming high-energy rays of the sunlight. A continuous layer of cells will provide the PBA with an energy-absorbing function, resulting in a non-heating, in other words cooling, effect at the bottom of the polymer. The above effect is utilized in moisture-resistant films and polymer coverings for sun-shielding purposes.

Finally there is the application example ranging from energy-absorbing cloth to perspiration-sensitive sports clothing which derives from the previous example. A PBA with a polymer component of polypropylene and a biocomponent of photosynthesizing organisms, such as a cyanobacterium or a unicellular alga type, is extruded to form a textile fiber. The temperature-resistant spores of the algae, after they have been extruded to form a fiber, are processed to produce a textile product. Use of fibers of this type in textile products ranges from covering fabrics, such as canvas, to sports clothing. For the absorption of moisture, for example sweat, the incorporated cells will convert the incident energy of sunlight into photosynthetic metabolites. As a result, the incident solar radiation is not converted into heat, but rather is extracted from the textile fiber, resulting in the desired cooling action. When drying out when no further sweat is being produced, the cells revert to their latent, inactive state. This is because the process is reversible.

Application examples relating to slow diffusion of cellular components and at least partially biomolecules in a moist environment are described below.

In a variant on the UV blocker from the above example, the active metabolite, astaxanthin, which very intensively blocks UV light, is incorporated in the PBA instead of the Haematococcus cells. As an alternative to the expensive component astaxanthin, it may be possible to use less expensive UV blockers. The diffusion rate of the UV blocker from the PBA in the middle layer of the polylamellar film to the periphery is regulated at a low to very low diffusion rate, depending on the quality and requirements. This fact is exploited in moisture-resistant UV-repellent films and polymer coverings, as well as for packaging material for food products. The polymer must in this case be durable and must not deteriorate in moist conditions.

In this context, an additional example of application consists in insect-repellent films, fibers-textile-and microgranules. This represents a variant on the above example. In this case, the bio component of the PBA is a bio-active organic molecule or a mixture of molecules, preferably substances, such as lipids, fatty acids, isoprene derivatives and hydrocarbons. In addition to a film or laminate, the processed product may also be a PBA which is processed to form a textile fiber or granule or microgranule, in which the biocomponent is released to the environment at a predetermined rate. This component has a specific repellent action to insects. Examples which have been tested include:

PBA with isoprene derivatives and/or branched hydrocarbons with a repellent activity to house dust mites. The PBA is extruded to form a textile fiber for weaving a fixed carpet and other products which have to be resistant to house dust mites; and

PBA with fatty acid components which are repellent to diptera, namely flies and mosquitoes, and biting and blood-sucking lice, Mallophaga, Anoplura, respectively, as well as the human head louse and poultry lice, cockroaches, ants and wasps. The PBA is granulated or extruded to form a textile fiber. The laden granules are mixed into the animal's coat, to protect against myiasis, horsefly and the like, or are scattered on the nesting site of the host of the parasite in question. Laden fibers are processed to form a protective textile as a nightcap to kill head lice, or what is known as a tissue with which an object can be rubbed to protect against ants, cockroaches, flies and the like.

Further to the above example, another important application is the use of the PBA's as a crop protection agent, in particular as a herbicide or even as a fungicide.

The biological activity of the organisms incorporated in the PBA gives the polymer new properties which were not previously known. The PBA ensures the desired environment for which the PBA was made, such as for example an anaerobic environment, complete oxygen barrier, energy absorption of solar radiation, controlled release of metabolites and the like.

The interaction and exchange of various types of organisms or microorganisms and/or molecules in the bio component of the PBA can also yield a large number of possible applications.

1-27. (canceled)

28. A method for producing polymer-bio aggregates comprising:
selecting a polymer;
selecting one or more organisms which are cells and/or cell products;
working said cells and/or said cell product into the polymer; thus
forming polymer-bio aggregates from said cells and/or cell products in said polymer-bio aggregates.

29. The method according to claim 28, wherein said working step is carried out under standard pressure conditions and a lower temperature limit of 100° C.

30. The method according to the claim 28, wherein said cells are at least one member selected from the group consisting of cysts, inactive cells or sleeping cells.

31. The method according to claim 28, wherein said cells are at least one member selected from the group consisting of prokaryotic cells, and eukaryotic cells.

32. The method according to claim 31, wherein said cells are of at least one member selected from the group consisting of protists, fungi, plant cells, and animal cells.

33. The method according to claim 28, wherein said cell products are metabolites.

34. The method according to claim 28, wherein said organisms are unicellular.

35. The method according to claim 28, wherein said organisms are multicellular.

36. The method according to claim 28, wherein said polymers are non-biodegradable polymers.

37. The method according to claim 36, wherein said polymers are polyolefins.

38. The method according to claim 36, wherein said polymers are polyethylenes.

39. The method according to the claim 38, wherein said polymer is PET.

40. The method according to claim 37, wherein said polymers are polypropylenes.

41. The method according to claim 28, wherein said polymers are polyesters.

42. The method according to claim 28, wherein said cells and/or cell products are embedded in said polymer.

43. The method according to claim 28, wherein said biopolymer is obtained from bringing up the cells and/or cell products while producing the polymer itself, wherein said biopolymer is obtained from a synthesis of said basis products.

44. The method according to claim 28, wherein said cells and/or cell products are thermally blended into an existing polymer.

45. The method according to claim 28, wherein said cells and/or cell products are blended in an existing polymer, and wherein said blending is performed warm as intermediate layer.

46. The method according to claim 30, wherein said cells and said polymer are agglomerated within a short period of time during which the polymer is liquid.

47. The method according to claim 28, wherein said cells and/or cell products are blended into an existing polymer, and wherein said blending is performed cold as intermediate layer.

48. A multilayer packaging material comprising a layer of a PBA produced according to claim 28 arranged as an intermediate layer.

49. The packaging material according to claim 48, wherein said polymer component of said PBA is composed of PET, and wherein said PBA comprises a type of yeast with a dry spore which is able to withstand the high temperatures of the production process.

50. The packaging material according to claim 49, wherein said PBA comprises an alga, and wherein the spores of said alga block UV-light.

51. A method of producing an oxygen barrier comprising:
producing a polymer-bio aggregate by the method of claim 28, and
producing a multi-layer food packaging material comprising said polymer-bio aggregate,
wherein said polymer-bio aggregate comprises spores that become activated to form respiring cells upon contact with water, and wherein said respiration of said cells withdraws all oxygen from inside said food packaging material.

52. A method for producing an insect repellent fiber or film comprising:
producing a polymer-bio aggregate by the method of claim 28, and
producing a fiber or film comprising said polymer-bio aggregate,
wherein said polymer-bio aggregate comprises one or more of a lipid, a fatty acid, an isoprene derivative or a hydrocarbon.

53. A method for producing a crop protection agent comprising:
producing a polymer-bio aggregate by the method of claim 28, and
producing a fiber or film comprising said polymer-bio aggregate, wherein said polymer-bio aggregate comprises a herbicide or a fungicide.

54. The method according to claim 31, wherein said prokaryote is a bacteria.
